SEQUENCE LISTING (2 sequences)

SEQ ID NO: 1 - protein, 331 aa, Streptomyces mobaraensis
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 2 - protein, 332 aa, Artificial Sequence
  Description of Artificial Sequence Synthetic polypeptide
  MOD_RES (1)..(1): M or absent
  MOD_RES (11)..(11): A, C or Q
  MOD_RES (15)..(15): D, H, L, M, N, W or Y
  MOD_RES (16)..(16): R, A, E or T
  MOD_RES (19)..(19): D, E or T
  MOD_RES (48)..(48): G or H
  MOD_RES (49)..(49): R or M
  MOD_RES (50)..(50): K, E or T
  MOD_RES (75)..(75): Q or C
  MOD_RES (135)..(135): N, S or T
  MOD_RES (137)..(137): A, C or S
  MOD_RES (138)..(138): L, K or V
  MOD_RES (148)..(148): L, E or M
  MOD_RES (165)..(165): E or F
  MOD_RES (170)..(170): P or E
  MOD_RES (171)..(171): F, I, L or V
  MOD_RES (200)..(200): S, A or G
  MOD_RES (300)..(300): S, A, E, K or V
XDSDDRVTPPXEPLXXMPXPYRPSYGRAETVVNNYIRKWQQVYSHRDXXXQQMTEEQREWLSYGCVGVTWVNSGXYPTNRLAFASFDEDRFKNELKNGRPRSGETRAEFEGRVAKESFDEEKGFQRAREVASVMXRXXENAHDESAYXDNLKKELANGNDALRNXDARSXXYSALRNTPSFKERNGGNHDPSRMKAVIYXKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWXEGYSDFDRGAYVITFIPKSWNTAPDKVKQGWP

(19) United States
(12) Patent Application Publication
(10) Pub. No.: US 2023/0167420 A1
(43) Pub. Date: Jun. 1, 2023

(54) TRANSGLUTAMINASE VARIANTS

(71) Applicant: Curie Co. Inc., Durham, NC (US)

(72) Inventors: Will SHINDEL, Durham, NC (US); Erika MILCZEK, Durham, NC (US)

(21) Appl. No.: 17/910,952

(22) PCT Filed: Mar. 10, 2021
(86) PCT No.: PCT/US2021/021766

§ 371 Date: Sep. 12, 2022

Related U.S. Application Data
(60) Provisional application No. 62/988,918, filed on Mar. 13, 2020.

(51) Int. Cl. C12N 9/10; A61Q 17/04; A61K 8/66; A01N 63/50

(52) U.S. Cl. CPC C12N 9/1044; A61Q 17/04; A61K 8/66; A01N 63/50; C12Y 203/02013

(57) ABSTRACT

Transglutaminase variants are disclosed. Applications of use for the variants, including use as preservatives, biocidal agents, and for modification of proteins, are disclosed.

### CROSS-REFERENCE TO RELATED APPLICATIONS

This application claims the benefit of U.S. Provisional Application No. 62/988,918, filed on Mar. 13, 2020, which is incorporated by reference herein in its entirety.

### FIELD OF THE INVENTION

The present invention relates to novel variants of Streptomyces mobaraensis transglutaminase. The variants may be used for conjugating proteins, peptides, or small molecules with increased specific activity and/or rates of crosslinking activity compared to wild-type Streptomyces mobaraensis transglutaminase. The variants may also be used as active biocidal enzymes and formulations thereof for use as agents for broad spectrum microbial control.

### BACKGROUND OF THE INVENTION

Transglutaminases (EC 2.3.2.13) are enzymes capable of catalyzing an acyl transfer reaction in which a γ-carboxy-amide group of a peptide bound glutamine residue is the acyl donor. Primary amino groups in a variety of compounds may function as acyl acceptors with the subsequent formation of monosubstituted γ-amides of peptide bound glutamine. When the ε-amino group of a lysine residue in a peptide chain serves as the acyl acceptor, the transglutaminases form intramolecular or intermolecular γ-glutamyl-ε-lysyl crosslinks.

Transglutaminase has found many applications in biotechnology and in the food processing industry, where it has earned the moniker “meat glue.” The peptide crosslinking activity has shown useful for a variety of industrial purposes ranging from food processing, biotechnology, pharmaceuticals, medical devices, personal and household goods, and leather and textile treatment.t

### SUMMARY OF THE INVENTION

Transglutaminase (Tgase) enzymes are provided herein. The enzymes are variants of Streptomyces mobaraensis Tgase (SEQ ID NO:1). Some of the variants demonstrate improvements in transamidation activity that is about 1.4-fold, about 1.6-fold, or 1.8-fold or greater than the wild-type Streptomyces mobaraensis enzyme (at least about or greater than about 40%, about 60%, or about 80% improvement in enzymatic activity).

It is desirable to have high specific activity of transglutaminase to allow for lower quantities of enzyme for crosslinking glutamine-donor substrates with amine substrates in the transamidation reaction, to allow for lower cost of product development. Additionally, it is beneficial to identify mutational variants of transglutaminase that exhibit higher initial rates to deliver shorter reaction times. One such example is in the case of preservation, where rapid crosslinking of proteins, e.g., cellular surface proteins, leads to superior microbial control, such as, but not limited to, faster or more effective microbial kill rate.

In one aspect, transglutaminase variant enzymes are provided. In some embodiments, the variant comprises or consists of an amino acid sequence selected from the sequences provided in Table 3, optionally further comprising an N-terminal methionine residue. In some embodiments, the transglutaminase is a variant of the mature transglutaminase enzyme of Streptomyces mobaraensis, selected from A10C or Q, D14H, L, M, N, W, or Y, R15A, E, or T, D18 E or T, G47H, R48M, K49E or T, Q74C, N134S or T, A136C or S, L137K, V, E, or M, E164F, P169E, F170I, L, or V, S199A or G, and S299A, E, K, or V, optionally further comprising an N-terminal methionine residue. In some embodiments, the transglutaminase is a circular permutant of any of the amino acid sequences depicted in Table 3, optionally further comprising an N-terminal methionine residue. In some embodiments, the transglutaminase enzyme further includes a pro-sequence.

In another aspect, methods are provided for increasing the shelf life of a product. The methods include incorporating a transglutaminase variant as described herein into the product in an amount effective to prevent or decrease growth of one or more microbe in comparison to an identical product that does not include the composition.

In another aspect, products are provided that include a transglutaminase variant as described herein in an effective amount to increase the shelf life of the product, in comparison to an identical product that does not include the enzyme. For example, the product may be a personal care, household, industrial, food, pharmaceutical, cosmetic, healthcare, marine, paint, coating, energy, plastic, packaging, or agricultural product. In some embodiments, the product bar soap, liquid soap, hand sanitizer, preoperative skin disinfectant, cleansing wipes, disinfecting wipes, body wash, acne treatment products, antifungal diaper rash cream, antifungal skin cream, shampoo, conditioner, cosmetics deodorant, antimicrobial creams, body lotion, hand cream, topical cream, aftershave lotion, skin toner, mouth wash, toothpaste, or sunscreen lotion. In other embodiments, the product is a wound care product selected from wound healing ointments, creams, and lotions, wound coverings, burn wound cream, bandages, tape, or steri-strips.

In another aspect, an enzyme composition is provided that includes: (i) a transglutaminase variant enzyme as described herein; and (ii) a substrate for the transglutaminase enzyme, such as a sunscreen molecule, a pigment, or a dye molecule. In some embodiments, the sunscreen molecule, pigment or dye molecule is conjugated to a molecule that includes a free amino group. For example, the molecule that includes a free amino group may be lysine, cadaverine, putrescine, hydrazine, adipic acid dihydrazide, sebacic dihydrazide, or hexamethylenediamine. In some embodiments, the sunscreen molecule, pigment, or dye molecule is conjugated to an amino acid, peptide, or protein with a free glutamine side chain. Cosmetic compositions that include the enzyme composition are also provided.

In another aspect, methods are provided for bonding color to a material or protein of interest. The methods include contacting the material or protein of interest with a transglutaminase variant enzyme as described herein and a pigment or dye molecule, wherein the transglutaminase variant enzyme is present in an amount effective to covalently bind the pigment or dye molecule to the material or protein of interest. In one embodiment, the protein of interest is a protein that is present in skin. For example, the protein that is present in skin may be collagen, keratin, and/or elastin.

In another aspect, products are provided that include a transglutaminase variant enzyme as described herein in an effective amount to add a color molecule onto a protein or a protein-, peptide-, or amino acid-containing material of interest when contacted with the product. In some embodiments, the product may be a personal care, cosmetic, leather, food, or agricultural product. Methods for modifying the color of a protein or material of interest are also provided, which include contacting the protein or material of interest with the product.

In another aspect, compositions are provided that include a transglutaminase variant enzyme as described herein in combination with one or more antimicrobial enzyme, peptide, or protein, wherein the composition possesses a preservative, biocidal, antimicrobial, or virucidal activity. In some embodiments, the antimicrobial enzyme, peptide, or protein is lysozyme, chitinase, lipase, lysin, lysostaphin, glucanase, DNase, RNase, lactoferrin, glucose oxidase, peroxidase, lactoperoxidase, lactonase, acylase, dispersin B, a-amylase, cellulase, nisin, bacteriocin, siderophore, polymyxin, or defensin.

In another aspect, a bacteriophage is provided, which includes a nucleic acid sequence that encodes a transglutaminase variant enzyme as described herein. In one embodiment, the composition provides antimicrobial activity. The composition may further include a pharmaceutically acceptable excipient.

### DESCRIPTION OF THE INVENTION

The Tgase crosslinking enzymes disclosed herein may be employed as biocidal agents for novel microbial control with applications in healthcare products, personal care or cosmetic formulations, packaging (e.g., food, cosmetic, and pharmaceuticals), textile and leather production, paints and coatings, and marine applications including water treatment and purification. In some embodiments, Tgase enzymes disclosed herein may be employed for permanently modifying proteins of interest, by way of example keratin and collagen, with dyes or proteins. In some embodiments, the Tgase enzymes may be used as preservatives.

Tgase enzymes that are mutant forms of the Streptomyces mobaraensis Tgase are disclosed herein. Specifically, the enzymes described herein are proteins obtained by mutating at least one amino acid in the polypeptide sequence of the wild-type Tgase, or circular permutants thereof, and observing transglutaminase transamidation activity between a glutamine amino acid residue and an amine (or hydroxylamine) acceptor.

Methods for recombinant expression of proteins with mutational substitutions have been described previously, for example, Molecular Cloning, A Laboratory Manual 4th ed., Cold Spring Harbor Press (1989), Current Protocols in Molecular Biology, John Wiley & Sons (1987-1997) and the like. Single point mutant enzymes can be generated using site-directed mutagenesis or any other methods known in the art. Such methods can include, but are not limited to, using kits and commercially available reagents such as the Kunkel method, KLD method, or Gapped duplex method, and examples of the kit, for example, QuickChange™ Site-Directed Mutagenesis Kit (Stratagene), GeneArt™ Site-Directed Mutagenesis System (Invitrogen), Q5©, Site-Directed Mutagenesis System (New England Biolabs), TaKaRa Site-Directed Mutagenesis System (Prime STAR® Mutagenesis Basal kit, or Muta-Direct™ Site Directed Mutagenesis Kit (iNtRON), and the like.

### I. Definitions

“A,” “an” and “the” include plural references unless the context clearly dictates otherwise.

The term “about” is used herein to mean plus or minus ten percent (10%) of a value. For example, “about 100” refers to any number between 90 and 110.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “amino acid” refers to a molecule containing both an amine group and a carboxyl group that are bound to a carbon, which is designated the alpha-carbon. Suitable amino 30 acids include, without limitation, both the D- and L-isomers of the naturally occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs.

As used herein, “antimicrobial” refers to a substance which is intended to kill or inhibit the growth of bacteria and viruses, according to the EPA.

The term “base pair” or “bp” as used herein refers to a partnership (i.e., hydrogen bonded pairing) of adenine (A) with thymine (T), or of cytosine (C) with guanine (G) in a double stranded DNA molecule. In some embodiments, a base pair may include A paired with Uracil (U), for example, in a DNA/RNA duplex.

As used herein, “biocide” refers to a substance which kills microorganisms, as defined by the U.S. Environmental Protection Agency (EPA).

A “circular permutant” refers to a protein that has a changed order of amino acids in its amino acid sequence in comparison with a reference sequence. The result is a protein structure with different connectivity, but overall similar three-dimensional (3D) shape in comparison to the reference protein.

The term “derived from” encompasses the terms “originated from,” “obtained from,” “obtainable from,” “isolated from,” “purified from,” and “created from,” and generally indicates that one specified material finds its origin in another specified material or has features that can be described with reference to another specified material.

The term “duplex” herein refers to a region of complementarity that exists between two polynucleotide sequences. The term “duplex region” refers to the region of sequence complementarity that exists between two oligonucleotides or two portions of a single oligonucleotide.

“Effective amount” as used herein refers to an amount (e.g., minimum inhibitory concentration (MIC)) of a preservative composition as disclosed herein that is sufficient to prevent or inhibit microbial growth. The preservative compositions of this patent are active against Gram positive bacteria, Gram negative bacteria, yeast, and/or mold.

As used herein, the term “expression” refers to the process by which a polypeptide is produced based on the nucleic acid sequence of a gene. The process includes both transcription and translation.

A “gene” refers to a DNA segment that is involved in producing a polypeptide and includes regions preceding and following the coding regions as well as intervening sequences (introns) between individual coding segments (exons).

“Household products” are products, other than personal care products, that would be used by individual consumers.

“Hybridization” and “annealing” refer to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may include two nucleic acid strands forming a duplex structure, three or more strands forming a multi-stranded complex, a single self-hybridizing strand, or any combination of these. A hybridization reaction may constitute a step in a more extensive process, such as the initiation of polymerase chain reaction (PCR), ligation reaction, sequencing reaction, or cleavage reaction, e.g., enzymatic cleavage of a polynucleotide by a ribozyme. A first nucleic acid sequence that can be stabilized via hydrogen bonding with the bases of the nucleotide residues of a second sequence is said to be “hybridizable” to the second sequence. In such a case, the second sequence can also be said to be 10 hybridizable to the first sequence. The term “hybridized” refers to a polynucleotide in a complex that is stabilized via hydrogen bonding between the bases of the nucleotide residues.

“Industrial products” refers to products that are used in industry.

The terms “isolated,” “purified,” “separated,” and “recovered” as used herein refer to a material (e.g., a protein, nucleic acid, or cell) that is removed from at least one component with which it is naturally associated, for example, at a concentration of at least 90% by weight, or at 15 least 95% by weight, or at least 98% by weight of the sample in which it is contained. For example, these terms may refer to a material which is substantially or essentially free from components which normally accompany it as found in its native state, such as, for example, an intact biological system. An isolated nucleic acid molecule includes a nucleic acid molecule contained in cells that ordinarily express the nucleic acid molecule, but the nucleic acid molecule is present extrachromosomally or at a chromosomal location that is different from its natural chromosomal location.

A “mature” polypeptide, protein or enzyme refers to the activated form of a zymogen or proprotein following cleavage of its pro-sequence or in the absence of the pro-sequence. In some embodiments, the mature enzyme may be produced as a separate polypeptide from the pro-sequence in order to eliminate a post-translational processing (activation) step.

The terms “microorganism” and “microbe” can include bacteria, protozoa, fungi, algae, amoebas, viruses, and molds life forms.

The term “mutation” herein refers to a change introduced into a parental sequence, including, but not limited to, substitutions, insertions, and deletions (including truncations), thereby producing a “mutant.” The consequences of a mutation include, but are not limited to, the creation of a new character, property, function, phenotype or trait not found in the protein encoded by the parental sequence.

The term “nucleotide” herein refers to a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ 30 or 5′ position of the pentose it is referred to as a nucleotide. A sequence of polymeric operatively linked nucleotides is typically referred to herein as a “base sequence,” “nucleotide sequence,” “polynucleotide sequence,” “oligonucleotide sequence”, or nucleic acid or polynucleotide “strand,” and is represented herein by a formula whose left to right orientation is in the conventional direction of 5′-terminus to 3′-terminus, referring to the terminal 5′ phosphate group and the terminal 3′ hydroxyl group at the “5′” and “3′” ends of the polymeric sequence, respectively.

“Optional” or “optionally” means that the subsequently described event, circumstance, or material may or may not occur or be present, and that the description includes instances where the event, circumstance, or material occurs or is present and instances where it does not occur or is not present.

As used herein, “pathogen” refers to microorganisms (e.g., bacteria, viruses, or parasites) that can cause disease in humans, animals, and/or plants.

“Peptide” refers to a compound consisting of two or more amino acids linked in a chain, the carboxyl group of each acid being joined to the amino group of the next by a bond of the type R—OC—NH—R′, for example, about 2 to about 50 amino acids.

The term “polymerase” herein refers to an enzyme that catalyzes the polymerization of nucleotides (i.e., the polymerase activity). The term polymerase encompasses DNA polymerases, RNA polymerases, and reverse transcriptases. A “DNA polymerase” catalyzes the polymerization of deoxyribonucleotides. An “RNA polymerase” catalyzes the polymerization of ribonucleotides. A “reverse transcriptase” catalyzes the polymerization of deoxyribonucleotides that are complementary to an RNA template.

The terms “polynucleotide,” nucleic acid,” and oligonucleotide” are used interchangeably. They refer to a polymeric form of nucleotides of any length, either deoxyribonucleotides or ribonucleotides, or analogs thereof. Polynucleotides may have any three-dimensional structure and may perform any function, known or unknown, may be single- or multi-stranded (e.g., single-stranded, double-stranded, triple-helical, etc.), and may contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. The following are nonlimiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may include modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise.

As used herein, “polypeptide” refers to a composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also, included within the definition are, for example, polypeptides containing one or more analogs of an amino acid (including, for example, unnatural amino acids, etc.), as well as other modifications known in the art.

As used herein, “preservative” is an agent added to a product as described to prevent (for some period of time) the growth of microorganisms, or the occurrence of undesirable chemical reactions (such as oxidation), that spoil or deteriorate, including deterioration of one or more utility, of the product.

A “promoter” refers to a regulatory sequence that is involved in initiating transcription of a gene by RNA polymerase. A promoter may be an inducible promoter or a constitutive 5 promoter. An “inducible promoter” is a promoter that is active under environmental or developmental regulatory conditions.

A “pro-sequence” refers to a polypeptide sequence within an expressed protein, e.g., a zymogen or proprotein, such as transglutaminase, which is typically cleaved from the protein to produce an active protein, such as an enzyme. In some embodiments, a pro-sequence may be essential for correct folding of the protein. In some embodiments, cleavage of the pro-sequence results in transition of an inactive enzyme to active enzyme.

The term “recombinant,” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally recombinant nucleic acids, polypeptides, and cells based thereon, have been manipulated such that they are not identical to related nucleic acids, polypeptides, and cells found in nature. A recombinant cell may also be referred to as “engineered.”

“Shelf life” refers to the length of time for which an item (e.g., a product as described herein) remains usable, fit for consumption, or saleable.

The phrases “substantially similar” and “substantially identical” in the context of at least two nucleic acids typically means that a polynucleotide includes a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) polynucleotide or polypeptide. Sequence identity may be determined using known programs such as BLAST, ALIGN, and CLUSTAL using standard parameters. (See, e.g., Altshul et al. (1990) J. Mol. Biol. 215:403-410; Henikoff et al. (1989) Proc. Natl. Acad. Sci. 89:10915; Karin et al. (1993) Proc. Natl. Acad. Sci. 90:5873; and Higgins et al. (1988) Gene 73:237). Software for performing BLAST analyses is publicly available through the National Center for Biotechnology Information. Also, databases may be searched using FASTA (Pearson et al. (1988) Proc. Natl. Acad. Sci. 85:2444-2448.) In some embodiments, substantially identical nucleic acid molecules hybridize to each other under stringent conditions (e.g., within a range of medium to high stringency). Nucleic acid “synthesis” herein refers to any in vitro method for making a new strand of polynucleotide or elongating an existing polynucleotide (i.e., DNA or RNA) in a template dependent manner. Synthesis, according to the invention, can include amplification, which increases the number of copies of a polynucleotide template sequence with the use of a polymerase. Polynucleotide synthesis (e.g., amplification) results in the incorporation of nucleotides into a polynucleotide (e.g., extension from a primer), thereby forming a new polynucleotide molecule complementary to the polynucleotide template. The formed polynucleotide molecule and its template can be used as templates to synthesize additional polynucleotide molecules. “DNA synthesis,” as used herein, includes, but is not limited to, polymerase chain reaction (PCR), and may include the use of labeled nucleotides, e.g., for probes and oligonucleotide primers, or for polynucleotide sequencing. “Under transcriptional control” is a term well understood in the art that indicates that transcription of a polynucleotide sequence depends on its being operably linked to an element which contributes to the initiation of, or promotes transcription.

Related (and derivative) proteins encompass “variant” proteins. Variant proteins differ from another (i.e., parental) protein and/or from one another by a small number of amino acid residues. A variant may include one or more amino acid mutations (e.g., amino acid deletion, insertion or substitution) as compared to the parental protein from which it is derived. Alternatively or additionally, variants may have a specified degree of sequence identity with a reference protein or nucleic acid, e.g., as determined using a sequence alignment tool, such as BLAST, ALIGN, and CLUSTAL (see, infra). For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence.

A “zymogen” or “proenzyme” refers to an inactive precursor of an enzyme, which may be converted into an active enzyme by catalytic action, such as via proteolytic cleavage of a pro-sequence.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

### II. Tgase Variants

Tgase variants with at least 1.4-fold (40%) improvement in enzyme activity, versus the wild-type enzyme from Streptomyces mobaraensis (SEQ ID NO:1), are disclosed herein.

Seq. Id. No. 1: Wild-Type mature TGase
DSDDRVTPPAEPLDRMPDPYRPSYGRAETVVNNYIRKWQQVYSHRDGRKQ
QMTEEQREWLSYGCVGVTWVNSGQYPTNRLAFASFDEDRFKNELKNGRPR
SGETRAEFEGRVAKESFDEEKGFQRAREVASVMNRALENAHDESAYLDNL
KKELANGNDALRNEDARSPFYSALRNTPSFKERNGGNHDPSRMKAVIYSK
HFWSGQDRSSSADKRKYGDPDAFRPAPGTGLVDMSRDRNIPRSPTSPGEG
FVNFDYGWFGAQTEADADKTVWTHGNHYHAPNGSLGAMHVYESKFRNWSE
GYSDFDRGAYVITFIPKSWNTAPDKVKQGWP

The amino acid sequences of examples of such variants, relative to the wild-type sequence set forth in SEQ ID NO:1, are disclosed in Table 3. For example, a Tgase variant with at least about 1.4-fold (40%) improvement in activity versus the wild-type enzyme may have any of the point mutations shown in Table 3.

Tgase variants herein may further include a pro-sequence. In some embodiments, the variant is expressed with a pro-sequence, either as part of the variant polypeptide sequence (e.g., an additional amino acid sequence as an extension of an amino acid sequence described in Table 3) or as a separate polypeptide. In some embodiments, the mature variant polypeptide is expressed in the presence of a polypeptide Tgase pro-sequence. In one embodiment, a DNA sequence that encodes the pro-sequence and the DNA sequence that encodes the mature Tgase variant are expressed as discrete polypeptide sequences from the same DNA template. In another embodiment, the DNA sequence that encodes the mature polypeptide is expressed from a first DNA template, and the DNA sequence that encodes the pro-sequence is expressed from a separate second DNA template. In another embodiment, the pro-sequence is synthesized chemically and added to an expression system prior to, during, or after expression of the mature polypeptide. In one example, the Tgase variant may be expressed in a cell free expression system, as disclosed in PCT Application No. US20/49226, which is incorporated by reference herein in its entirety.

In some embodiments, the Tgase variant is expressed, e.g., expressed recombinantly, with a homologous pro-sequence, i.e., the native pro-sequence for the Tgase enzyme, i.e., the pro-sequence for the wild-type Tgase enzyme from the same organism. In other embodiments, the Tgase variant is expressed, e.g., expressed recombinantly, with a heterologous pro-sequence, i.e., a pro-sequence for the same enzyme but from a different organism or a pro-sequence for a different enzyme from the same or different organism.

The mature wild-type Streptomyces mobaraensis Tgase enzyme lacks the N-terminal methionine residue encoded by the gene sequence that encodes the enzyme. In some embodiments, the Tgase variant is expressed as a variant of the mature Streptomyces mobaraensis Tgase without an N-terminal methionine residue. In other embodiments, the Tgase is expressed as the mature Tgase with an additional N-terminal methionine residue, which may be provided by an expression vector from which the Tgase is expressed.

In some embodiments, a Tgase variant may be a circular permutant of a Tgase enzyme, e.g., a circular permutant of the wild-type Tgase enzyme (SEQ ID NO:1) or of a Tgase variant described herein (e.g., a variant described in Table 3). In some embodiments, the Tgase variant may be a circular permutant of a Tgase variant as described in Table 1, optionally further including an N-terminal methionine residue. The circular permutants may provide novel substrate specificities, product profiles, and reaction kinetics versus the parent enzyme, i.e., the wild-type enzyme or a disclosed variant, e.g., as depicted in Table 3. A circular permutant retains the same basic folding of the parent enzyme, but has the N-terminus in a different position, with the original N- and C-termini connected, optionally by a linking sequence. In a Tgase wild-type or variant circular permutant, the N-terminal residue of the wild-type or variant enzyme is positioned at a site in the protein other than the natural N-terminus.

### III. Antimicrobial Compositions

Disclosed are compositions, e.g., biocidal, preservative, antimicrobial, anti-bacterial, and anti-viral (virucidal) compositions that include one or more Tgase variant enzyme as described herein, such as any of the variants disclosed in Table 3, optionally with an N-terminal methionine residue, including circular permutants thereof, and optionally with a pro-sequence as described herein. Such a composition may be included in or with (e.g., within or associated with) products to be preserved, e.g., for microbial control. The Tgase variant enzyme may catalyze a reaction of amino acid residues on a protein, thereby effecting, for example, protein cross-linking or binding a molecule of interest to a protein.

In some embodiments, the compositions include one or more Tgase variant enzyme, e.g., comprising or consisting of one or more Tgase variant as disclosed herein, in an amount effective to inhibit microbial (e.g., bacterial) growth, e.g., inhibition of 80% to 100%, or any of at least about 80%, 85%, 90%, 95%, 98%, or 99% of microbial growth, in a product to be preserved.

Preservatives are antimicrobial ingredients added to product formulations to maintain the microbiological safety of the products by inhibiting the growth of and reducing the amount of microbial contaminants. US Pharmacopeia has published protocols for acceptable microbial survival for preservatives in cosmetics and personal care products. These tests include USP 51 (Antimicrobial Effectiveness Test) and USP 61 (Microbial Limits Test) (https://www.fda.gov/files/about%20fda/published/Pharmaceutical-Microbiology-Manual.pdf).

The effectiveness of the preservative system disclosed herein is determined based on the MIC (minimum inhibitory concentration) against a variety of microbes, including, but not limited to, Gram positive bacteria, Gram negative bacteria, yeast and/or mold (e.g. E. coli DH10ß, E. coli ATCC 8739, B. subtilis BGSC 1A976, C. albicans ATCC 10231, and/or A. brasiliensis ATCC 16404). Minimum inhibitory concentrations (MICs) are defined as the lowest concentration of an antimicrobial that will inhibit the growth of a microorganism. Microbial growth may be determined, for example, by spectrophotometric methods (the optical density at 600 nm) or with a cell viability assay (BacTiter Glo, Promega).

In some embodiments, the compositions include one or more additional biocidal enzymes, such as a cross-linking enzyme, nuclease, hydrolase, protease, and/or lytic enzyme. In some embodiments, the composition further includes one or more biocidal chemical, such as, but not limited to, chitosan, polylysine, and/or quaternary ammonium compounds. Exemplary, but nonlimiting examples of biocidal enzymes, compositions, and formulations, and methods of use thereof, are disclosed in PCT/US20/21211, which is incorporated by reference herein in its entirety.

Without wishing to be bound by theory, the use of a biocidal enzyme enhances the antimicrobial properties of a biocidal chemical by providing an additional mechanism of antimicrobial action. Chitosan, for example, ruptures the cell membrane and leads to spillage of the cell contents. The cross-linking Tgase enzyme can cross-link proteins vital for cell function both on the surface of the cell and within the cell. This combination of both materials together reduce the quantity of the materials needed and provide additional stability to the enzyme allowing for greater activity over time (less chitosan and less enzyme) and reduce the undesirable effects that may accompany the use of biocidal chitosan.

A. Biocidal Proteins and Peptides

In some embodiments, the compositions include one or more antimicrobial peptides. Examples of antimicrobial peptides include, but are not limited to, nisin and pediocin.

In some embodiments, the compositions include one or more antimicrobial proteins. Examples of antimicrobial proteins include, but are not limited to, casein.

Nonlimiting examples of known biocidal enzymes and antimicrobial peptides, which may be included in combination with a Tgase variant enzyme as disclosed herein, are shown in Table 1. In some embodiments, a Tgase variant enzyme as described herein may be utilized in a biocidal, preservative, anti-bacterial, or anti-viral (virucidal) composition in combination with one or more of the antimicrobial enzymes, peptides, or proteins described in Table 1.

TABLE 1
Enzymes, Peptides and Proteins with Known Antimicrobial Properties

| Mechanism | Enzyme | Description | Citation |
|---|---|---|---|
| Lytic | Lysozyme | Produced by animals as part of the | Ibrahim et al. (2001) |
| | | innate immune system. | FEBS Letters |
| | | Hydrolyzes the peptidoglycan | 506(1): 27-32; |
| | | subunits in the bacterial cell wall. | Małaczewska et al. (2019) |
| | | | BMC Vet. Res. 15: 318 |
| | Chitinase | Secreted by soil bacteria including | Martínez-Zavala et al |
| | | Bacillus thuringiensis to combat | (2020) Front. Microbiol. |
| | | insects and fungi | 10: 3032 |
| | Lipase | Hydrolyzes extracellular lipids and | Prabhawathi et al. |
| | | polymers. | (2014) PLoS One 9(5) |
| | Lysin | Utilized by bacteriophages to | Hoops et al. (2008) Appl. |
| | | hydrolyze the glycan component of | Environ. Microbiol. |
| | | bacterial cell wall | 75: 5, 1388-1394 |
| | Lysostaphin | Metalloendopeptidase which | Kokai-Kun et al. (2003) |
| | | cleaves the pentaglycine bridges | Antimicrob Agents |
| | | found in cell wall peptidoglycan. | Chermother |
| | | | 47(5): 1589-1597 |
| | Glucanase | Secreted by soil bacteria including | Shafi et al. (2017) |
| | | Bacillus species to degrade the | Biotechnology & |
| | | fungal cell wall. Has also been | Biotechnological |
| | | utilized as an algicide and for | Equipment 31: 3 446-459 |
| | | biofilm control. | |
| Nuclease | DNase | Hydrolyzes extracellular nucleic | Kaplan et al. (2012) |
| | | acids and viral genomic DNA. | J. Antibiot. (Tokyo) |
| | | | 65(2): 73-77 |
| | RNase | Hydrolyzes viral RNA. | Wirth (1992) |
| | | | WO1994000016A1 |
| | Lactoferrin | Sequesters essential iron ions to | Niaz et al. (2019) |
| | | prevent microbial growth. Also | International Journal of |
| | | possesses nuclease activity and | Food Properties 22: 1 |
| | | hydrolyzes biofilm polymers. | 1626-1641 |
| Oxidoreductase | Glucose Oxidase | Oxidizes glucose to D-glucono-δ- | Wong et al. (2008) Appl |
| | | lactone and hydrogen peroxide. | Microbiol Biotechnol. |
| | | | 78(6): 927-938 |
| | Peroxidase | Oxidizes inert substrates to form | Ihalin et al. (2006) Arch. |
| | | biocidal actives. | Biochem. Biophys. 445, |
| | | | 261-268 |
| | Lactoperoxidase | Oxidizes inert substrates to form | White et al. (1983) |
| | | biocidal actives. | Antimicrob Agents |
| | | | Chemother 32(2): 267-272 |
| Quorum | Lactonase | Hydrolyzes quorum sensing | Schwab et al. (2019) |
| Quenching | | lactones, preventing activation of | Front Microbiol. 10: 611 |
| | | biofilm- and pathogenesis- | |
| | | promoting pathways. | |
| | Acylase | Hydrolyzes quorum sensing | Vogel et al. (2020) |
| | | lactones, preventing activation of | Front. Chem. 8: 54 |
| | | biofilm- and pathogenesis- | |
| | | promoting pathways. | |
| Hydrolase | Dispersin B | Hydrolyzes biofilm polymers | Izano et al. (2007) J |
| | | | Dent Res 86(7): 618-622 |
| | α-amylase | Hydrolyzes extracellular | Craigen et al. (2011) |
| | | polysaccharides. | Open Microbiol J. 5: 21-31 |
| | Cellulase | Hydrolyzes the cellulose | Loiselle et al. (2003) |
| | | component of biofilms and algal | Biofouling 19(2): 77-85 |
| | | cell walls. | |
| Antimicrobial | Nisin | Increases permeability of the | Li et al. (2018) Appl |
| Peptides | | microbial cell membrane. | Environ Microbiol 18(12) |
| | Bacteriocin | Modes of action include inhibition | Meade et al. (2020) |
| | | of cell wall synthesis and | Antibiotics 9(1):32 |
| | | increasing cell membrane | |
| | | permeability. | |
| | Siderophore | Binds to and sequesters iron ions | Raaska et al. (1999) J |
| | | | Indust Microbiol |
| | | | Biotechnol 22, 27-32 |
| | Polymyxin | Increases permeability of the | Poirel et al. (2017) Clin |
| | | microbial cell membrane. | Microbiol Rev 30: 577-596 |
| | Defensin | Increases permeability of the | Gans (2003) Nat Rev |
| | | microbial cell membrane. | Immunol 3, 710-720 |

B. Biocidal Chemicals

In some embodiments, a Tgase variant as described herein may be formulated with one or more biocidal chemical, including, but not limited to chitosan, polylysine, or quaternary ammonium compounds, for example, for use as a biocidal, preservative, anti-bacterial, or anti-viral (virucidal) composition. Nonlimiting examples of biocidal chemicals are shown in Table 2.

TABLE 2
Examples of Biocidal Chemicals for Antimicrobial Applications

| Classification | Chemical |
|---|---|
| Polymers | Chitosan |
| | N,N,N-trimethyl chitosan |
| | ε-poly-lysine |
| | Polyvinylbenzyl-dimethylbutyl ammonium chloride |
| | Polyvinylbenzyl trimethyl ammonium chloride |
| | Quaternary ammonium polyethyleneimine |
| | Quaternary phosphonium modified epoxidized natural |
| | rubber |
| | Arginine-tryptophan-rich peptide |
| | Guanylated polymethacrylate |
| | Ammonium ethyl methacrylate homopolymers |
| | Metallo-terpyridine carboxymethyl cellulose |
| | Poly(n-vinylimidazole) modified silicone rubber |
| Quaternary | Cocoamidopropyl Betaine |
| Ammonium | Myristamidopropyl-pg-dimonium Cl Phosphate |
| | Benzalkonium Chloride |
| | Quaternium-6 |
| | Coco Betaine |
| Detergents | Sodium Lauryl Sulfate |
| | Dodecylbenzenesulfonic Acid |
| Chaotropic | Polyamidopropyl biguanide |
| Agent | Guanidinium chloride |
| Organic Acids | Lactic Acid |
| | Citric Acid |
| | Salicylic Acid |
| | Sorbic Acid |
| | Acetic Acid |
| | Dehydroacetic Acid |
| | Peracetic Acid |
| | Benzoic Acid |
| Phenols & | Ethanol |
| Alcohols | Isopropanol |
| | Dichlorobenzyl Alcohol |
| | Glycerol |
| | Caprylyl Glycol |
| | Ethylhexylglycerin |
| | Benzyl Alcohol |
| | 2-Phenoxyethanol |
| Aldehydes & | Glutaraldehyde |
| Aldehyde | Formaldehyde |
| Releasers | Sodium Hydroxymethylglycerate |
| | DMDM Hydantoin |
| Base | Sodium Hydroxide |
| Oxidizers | Hydrogen Peroxide |
| Parabens | Methyl Paraben |
| | Ethyl Paraben |
| | Propyl Paraben |
| Misc | Natamycin |
| | Benzisothiazolinone |
| | Bronopol |
| | Sorbitan Caprylate |
| | Ethyl Lauroyl Arginate |
| | Methylisothiazolinone |
| | Cetylpyridinium Chloride |
| | Chlorphenesin |
| | Zinc Omamide |
| | Sodium Omamide |
| | N-(3-aminopropyl)-N-dodecylpropane- |
| | 1,3-diamine |
| | Methylchloroisothiazolinone |
| | 2,2-dibromo-3-nitrilopropionamide |
| | 1-Octadecanaminium, N,N-dimethyl-N-[3- |
| | (trimethoxysilyl)propyl]-, chloride |
| | Saponin |
| | Sodium Benzoate |

1. Quaternary Ammonium Compounds

Quaternary ammonium compounds containing biopolymers, like chitosan and its more acetylated form chitin, are well known for their antimicrobial activity (Kong, et al. (2010) Int. J. of Food Microbiol. 144: 51-63). The antimicrobial activity of chitin, chitosan and their derivatives against different groups of microorganisms, such as bacteria, yeast, and fungi, is known.

Quaternary ammonium compounds (non-limiting examples include, cetyl pyridinium chloride, benzethonium chloride, benzalkonium chloride, polyaminopropyl biguanide), have limited use for personal care industry due to specific incompatibilities with other cosmetic ingredients.

Lonza's Geogard series of preservative blends avoids use of parabens in their new creations (Geogard 233S, Geogard 233S, Geogard 233S, Geogard 361) however, these antimicrobial compositions are based on cationic benzethonium chloride which gets deactivated by many anionic ingredients that form important part of topical personal care formulations.

2. Aldehydes & Aldehyde-Releasing Compounds

Formaldehyde is classified as Category 3 CMR (carcinogenic, mutagenic and reproductive toxicity). However, it is interesting to note that a few antimicrobials that slowly release formaldehyde are still being used and being commercially manufactured. Due to the paucity of effective and well-accepted antimicrobials, the industry is forced to continue with the use of formaldehyde donors like DMDM hydantoin (CAS 6440-58-0), imidazolidinyl urea, and diazolidinyl urea (CAS 39236-46-9). The formaldehyde released by these substances is capable of reacting with several cosmetic ingredients via its very reactive aldehydic carbonyl functionality. For example, the only available and globally approved UV-A absorber, Avobenzone, reacts with formaldehyde that is released by formaldehyde derivatives. This is a significant disadvantage for sunscreen formulations. Preservative blends, Clariant's Niapaguard PDU and Cognis's Elestab 305, ISP's Germaben II, Germaben H-E, exploit combinations of parabens with diazolidinyl urea. ISP's Germall Plus and Lonza's Glydant Plus utilize diazolidinyl urea along with iodopropynyl butyl carbamate (IPBC). McIntyre's Paragon series has DMDM hydantoin and other antimicrobials like paraben, phenoxy ethanol and IPBC. Symrise's Neo-Dragocide and Thor's Microcare IMP exploit synergy between parabens and imidazolidinyl urea.

3. Parabens

Parabens are esters of p-hydroxy benzoic acid. Paraben compounds include in particular Methyl-paraben (CAS 99-76-3), Ethyl-paraben (CAS 120-47-8), Propyl-paraben (CAS 94-13-3), Butyl-paraben (CAS 94-26-8), Isopropyl-paraben (CAS 4191-73-5), and Benzyl-paraben (CAS 94-18-8). Clariant's ‘Phenonip’ is a blend of six antimicrobials out of which the five are parabens. The same company offers blends of only parabens as ‘Nipastat’ and ‘Nipasept’, Cognis's Elestab FL 15, Elestab 48, Elestab 50J, Elestab 305, Elestab 388, Elestab 3344, Elestab 4112, Elestab 4121, Elestab 4150 Lipo are all blends of antimicrobials with at least one paraben in them. Induchem's Uniphen P23, ISP's Germaben and LiquaPar series of blends contain several parabens. Galaxy Surfactants offers Galguard NK1 and Galguard NK2 blends that are based on four and five paraben blends respectively with phenoxy ethanol. Five blends by McIntyre/Rhodia from their ‘Paragon’ series have several parabens. Neolone MXP of Rohm and Haas has parabens with methyl isothiazolinone. Neo-Dragocide series of blends from Symrise has parabens. Euxyl K 300 of Schulke and Mayr has five parabens. Thor's Microcare PM4 and Microcare PM5 have four and five parabens respectively. Parabens are phenol derivatives; all phenolic antimicrobials have phenolic ‘hydroxyl’ group and that is a very reactive organic functionality with very acidic hydrogen with pKa of 10.

4. Halogenated Compounds

Nalco's Merguard series (four blends) relies on halogenated molecules, methyl dirbromo glutaronitrile and 2-bromo-2-nitro-1,3-diol. Several blends of Euxyl series from Schulke and Mayr are based on chlorothiazolinones, methyl dibromo glutaronitrile, 2-bromo-2-nitro-1,3-diol and diazolidinyl urea. Microcare series from Thor employs parabens, 2-bromo-2-nitro-1,3-diol, iodopropynyl butylcarbamate (IPBC), imidazolidynyl urea, and diazolidinyl urea.

The other examples of halogenated antimicrobials are chlorphenesin, and chlorhexidine. It is common knowledge that like phenolic compounds, the halogenated organic molecules exhibit significant levels of toxic effects. For example, IPBC has risk of thyroid hormonal disturbances due to its iodine content. It has not been allowed in Japan and in the EU is allowed only up to 0.02% in leave-on products. Similarly, the EU permits usage of methyl dirbromo glutaronitrile only up to 0.1% in rinse-off products only. Bronopol, 2-bromo-2-nitropropane-1,3-diol, is implicated in generation of carcinogenic nitrosoamines on interacting with some of the nitrogen containing cosmetic ingredients. The antimicrobial efficacy of methyl chloro isothiazolinone is so powerful that it is allowed only in rinse-off products at 15 ppm concentration. Chloromethyl isothiazolinone does have a very broad spectrum of anti-microbial activity, but the toxicity of such powerful anti-microbials is extremely high and hence cosmetic formulators do not prefer to use this kind of powerful antimicrobial in the cosmetics that remain on human skin for a long time. It is reasonable to expect that any strong bactericide at a low concentration (ppm level) is likely to be equally lethal to any other cells of a living organism, including human cells. This is the precise reason why in Japan chloromethyl isothiazolinone is not allowed for preservation if the product is going to come in contact with the mucous membrane.

Halogenated compounds include 2,4-dichlorobenzyl-alcohol, Chloroxylenol (also known as 4-chloro-3,5-dimethyl-phenol, Bronopol (also known as 2-bromo-2-nitropropane-1,3-diol, iodopropynyl butyl carbamate.

C. Vector Delivery

The compositions described herein may include vectors (e.g., bacteriophage), for the delivery of genetic material encoding one or more biocidal enzyme(s) as described herein.

As used herein, “bacteriophage” and “phage” are used interchangeably to refer to a bacteriophage isolate in which members of the isolate have substantially the same genetic makeup, such as sharing at least about any of 90%, 95%, 99%, 99.9% or more sequence identity in the genome. “Bacteriophage” or “phage” refers to the parent bacteriophage as well as the progeny or derivatives (such as genetically engineered versions) thereof. The bacteriophage can be a naturally occurring phage isolate, or an engineered phage, including vectors, or nucleic acids that encode at least all essential genes, or the full genome of a phage to carry out the life cycle of the phage inside a host bacterium.

### IV. Products

Products disclosed herein include personal care products, household products, industrial food, pharmaceutical, cosmetic, healthcare, marine, paints, coatings, adhesives, energy, plastic, packaging, or agricultural products, which include an effective amount, for example, about 0.0001% w/v to about 5% w/v, of a Tgase variant enzyme as described herein, or a composition thereof as described herein, to act as an antimicrobial agent, e.g., preservative, in the product.

In some embodiments, one or more Tgase variant is included in a personal care product, such as, but not limited to, bar soap, liquid soap (e.g., hand soap), hand sanitizer (including rinse off and leave-on alcohol based and aqueous-based hand disinfectants), preoperative skin disinfectant, cleansing wipes, disinfecting wipes, body wash, acne treatment products, antifungal diaper rash cream, antifungal skin cream, shampoo, conditioner, cosmetics (including but not limited to liquid or powder foundation, liquid or solid eyeliner, mascara, cream eye shadow, tinted powder, “pancake” type powder to be used dry or moistened, make up removal products, etc.), deodorant, antimicrobial creams, body lotion, hand cream, topical cream, aftershave lotion, skin toner, mouth wash, toothpaste, sunscreen lotion, and baby products such as, but not limited to, cleansing wipes, baby shampoo, baby soap, and diaper cream. In some embodiment, one or more Tgase variant is included in a wound care item, such as, but not limited to, wound healing ointments, creams, and lotions, wound coverings, burn wound cream, bandages, tape, and steri-strips, and medical articles such as medical gowns, caps, face masks, and shoe-covers, surgical drops, etc. In some embodiments, one or more Tgase variant is included in an oral care product, such as mouth rinse, toothpaste, or dental floss coating, a veterinary or pet care product, a preservative composition, or a surface disinfectant, such as a disinfectant solution, spray or wipe.

In some embodiments, one or more Tgase variant is incorporated into a household or industrial product, for example, as a preservative substance. For example, the Tgase variant(s) may be included in a household cleaner, such as concentrated a liquid cleaner or spray cleaner, cleaning wipes, dish washing liquid, dish washer detergent, spray-mop liquid, furniture polish, indoor paint, outdoor paint, dusting spray, laundry detergent, fabric softener, rug/fabric cleaner, window and glass cleaner, toilet bowl cleaner, liquid/cream cleanser, etc. In some embodiments, one or more Tgase variant may be included in a food wash product, e.g., designed to clean fruits and vegetables prior to consumption, packaging, and food coatings.

Other products into which Tgase variants as described herein may be incorporated include, but are not limited to, food, pharmaceutical, cosmetic, healthcare, marine, paint, coating, energy (e.g., fracking fluid), plastic, packaging, and agricultural products. In some embodiments, the Tgase variant may be incorporated into HVAC systems, cooling ponds, water purification systems, or may be used in an industrial application, such as, but not-limited to, pulp and paper processing.

Products disclosed herein include cosmetics and personal care products which include a Tgase variant or composition thereof as described herein, and one or more color producing molecule, in an amount effective to bond color to a surface, such as covalently binding to one or more protein of skin, e.g., collagen, keratin, and/or elastin, or to a protein of a food product, such as an edible casing for a processed food product, e.g., a sausage casing. In some embodiments, an effective amount of the Tgase variant enzyme is up to about 1% w/v.

In some embodiments, a product or composition which includes Tgase variant as described herein, further includes one or more additional enzymes selected from acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannases, esterases, exo-mannanases, galactanases, glucoamylases, hemicellulases, hyaluronidases, keratinases, laccases, lactases, ligninases, lipases, lipoxygenases, mannanases, oxidases, pectate lyases, pectin acetyl esterases, pectinases, pentosanases, peroxidases, phenoloxidases, phosphatases, phospholipases, phytases, polygalacturonases, beta-glucanases, tannases, xylan acetyl-esterases, xylanases, xyloglucanases, xylosidases, metalloproteases, serine proteases, or combinations thereof.

In some embodiments, a Tgase variant enzyme, such as any of the variants disclosed in Table 3, optionally with an N-terminal methionine residue, including circular permutants thereof, and optionally with a pro-sequence as described herein, or a composition thereof as described herein, is included as an antimicrobial agent in any of the products disclosed herein at a concentration of any of at least about 0.0001% w/v, 0.0005% w/v, 0.001% w/v, 0.005% w/v, 0.01% w/v, 0.05% w/v, 0.1% w/v, 0.5% w/v, 1% w/v, 1.5% w/v, 2% w/v, 2.5% w/v, 3% w/v, 3.5% w/v, 4% w/v, 4.5% w/v, or 5% w/v. In some embodiments, the Tgase variant enzyme of composition thereof is included at a concentration of any of about 0.0001% w/v to about 0.0005% w/v, about 0.001% w/v to about 0.005% w/v, about 0.005% w/v to about 0.01% w/v, about 0.01% w/v to about 0.05% w/v, about 0.05% w/v to about 0.1% w/v, about 0.1% w/v to about 0.5% w/v, about 0.5% w/v to about 1% w/v, about 1% w/v to about 1.5% w/v, about 1.5% w/v to about 2% w/v, about 2% w/v to about 2.5% w/v, about 2.5% w/v to about 3% w/v, about 3% w/v to about 3.5% w/v, about 3.5% w/v to about 4% w/v, about 4% w/v to about 4.5% w/v, about 4.5% w/v to about 5% w/v, about 0.0001% w/v to about 0.001% w/v, about 0.001% w/v to about 0.01% w/v, about 0.01% w/v to about 0.1% w/v, about 0.1% w/v to about 1% w/v, about 1% w/v to about 2.5% w/v, about 2.5% w/v to about 5% w/v, or about 1% w/v to about 5% w/v.

In some embodiments, products in which a Tgase variant enzyme or composition thereof as described herein is included as an antimicrobial agent do not include a petrochemically derived preservative substance, such as, but not limited to, parabens, formaldehyde and formaldehyde releasers, isothiazolinones, phenoxyethanol, and/or organic acids (such as sodium benzoate). In some embodiments, a Tgase variant enzyme as described herein, alone or in combination with a biocidal chemical, e.g., chitosan, is the only antimicrobial, e.g., antibacterial or preservative, agent in the product. In some embodiments, a Tgase variant enzyme as described herein is included as an antimicrobial agent in combination with one or more additional antimicrobial agent(s), such as, but not limited to, one or more petrochemically derived preservative substance(s). In some embodiments, a Tgase variant enzyme as described herein is included as an antimicrobial agent in combination with one or more additional antimicrobial agent(s), such as, but not limited to, one or more petrochemically derived preservative substance(s).

In some embodiments, preservative blends are compatible with products, stable towards oxidizing or reducing agents and to normal range of pH (4.5 to 8.0) of various products.

Non-limiting examples of products in which the Tgase variants described herein may be incorporated are described in PCT Application No. PCT/US20/21211, and in U.S. Provisional Application Nos. 63/010,987, 63/074,288, and 63/075,763, which are incorporated herein by reference in their entireties.

A. Personal Care Products

An Tgase variant enzyme or composition thereof, e.g., preservative composition, as described herein can be incorporated into any personal care product. Personal care products into which the disclosed Tgase variant enzymes compositions may be incorporated include, but are not limited to, bar soap, liquid soap (e.g., hand soap), hand sanitizer (including rinse off and leave-on alcohol based and aqueous-based hand disinfectants), preoperative skin disinfectant, cleansing wipes, disinfecting wipes, body wash, acne treatment products, antifungal diaper rash cream, antifungal skin cream, shampoo, conditioner, cosmetics (including but not limited to liquid or powder foundation, liquid or solid eyeliner, mascara, cream eye shadow, tinted powder, “pancake” type powder to be used dry or moistened, make up removal products, etc.), deodorant, antimicrobial creams, body lotion, hand cream, topical cream, aftershave lotion, skin toner, mouth wash, toothpaste, sunscreen lotion, and baby products such as, but not limited to, cleansing wipes, baby shampoo, baby soap, and diaper cream. The present subject matter may also be applied to wound care items, such as, but not limited to, wound healing ointments, creams, and lotions, wound coverings, burn wound cream, bandages, tape, and steri-strips, and medical articles such as medical gowns, caps, face masks, and shoe-covers, surgical drops, etc. Additional personal care products include, but are not limited to, oral products such as mouth rinse, toothpaste, dental floss coatings, veterinary and pet care products, preservative compositions, and surface disinfectants, including solutions, sprays or wipes.

In general, a Tgase variant enzyme c as disclosed herein can be incorporated into any suitable personal care product intended for use in modifying the appearance of skin, such as a cosmetic product (e.g., lipstick, foundation, blush, or eye makeup). Cosmetic products into which the disclosed compositions may be incorporated include, but are not limited to, liquid or powder foundation, liquid or solid eyeliner, blush, eye shadow, tinted powder, “pancake” type powder to be used dry or moistened, lip color, or makeup setting sprays, etc. The disclosed compositions may also be incorporated into a bronzer or artificial tanning product. Additionally, the disclosed compositions may be incorporated into a sunscreen product, such as a chemical sunscreen, e.g., to bind a sunscreen chromophore (such as, but not limited to, oxybenzone, avobenzone, octisalate, octocrylene, homosalate, or octinoxate, or a derivative thereof) to skin protein.

In some embodiments, the personal care products that are protected from the microbial contamination by the disclosed enzymes and compositions can be of any type of such as emulsions, gels, serums, solutions, toners, lotions, creams, spray, gel, powder, stick and cleansers.

The personal care product formulation typically includes a base formulation to which the preservative composition of the present disclosure is added. The base formulation may contain numerous and different ingredients depending upon the end use application. The personal care product formulation, for instance, may contain solvents, surfactants, emulsifiers, consistency factors, conditioners, emollients, skin care ingredients, moisturizers, thickeners, lubricants, fillers, antioxidants, other preservatives, active ingredients, in particular dermatologically active ingredients, fragrances and the like, as well as mixtures thereof. Active ingredients as mentioned herein include, for example, anti-inflammatories, and optionally, anti-bacterials, antifungals and the like agents. Active ingredients suited for topical applications are particularly preferred.

In some embodiments, the personal care product does not contain any additional preservatives, such as a petrochemical derived preservative substance. In some embodiments, the personal care product includes one or more additional preservative substance, such as a petrochemical derived preservative, in addition to the enzyme or enzyme/polymer composition described herein.

In some embodiments, the personal care product does not include conventional anti-bacterial and/or antifungal “active agents” that are typically included in personal care products. Conventional anti-bacterials used in hand soap include: Cloflucarban, Fluorosalan, Hexachlorophene, Hexylresorcinol, Iodine complex (ammonium ether sulfate and polyoxyethylene sorbitan monolaurate), Iodine complex (phosphate ester of alkylaryloxy polyethylene glycol), Nonylphenoxypoly (ethyleneoxy) ethanoliodine, Poloxamer-iodine complex, Povidone, Undecoylium chloride iodine complex, Methylbenzethonium chloride, Phenol, Phenol 16, Secondary amyltricresols, Sodium oxychlorosene, Tribromsalan, Triclocarban, Triclosan, and Triple dye. Conventional antimicrobials used as preservatives in consumer product formulations include: parabens, formaldehyde and formaldehyde releasers, isothiazolinones, phenoxyethanol, and organic acids (such as sodium benzoate).

In some embodiments, a Tgase variant enzyme as described herein, alone or in combination with (e.g., blend) a biocidal chemical, including but not limited to, chitosan, is the only antibacterial, antifungal, antimicrobial, or preservative agent in the product. In some embodiments, the Tgase variant enzyme, alone or in combination (e.g., blend) a biocidal chemical, such as but not limited to, chitosan, is combined with one or more additional preservative substance, such as one or more petrochemically derived preservative substance. In some embodiments, one or more biobased preservative (i.e., Tgase variant enzyme or composition thereof as disclosed herein) is combined with one or more synthetic preservative (e.g., petrochemical derived substance) and the preservative (e.g., antimicrobial) effect achieved between the biobased and synthetic preservatives is additive or synergistic. In some embodiments, one or more biobased preservative (i.e., Tgase variant enzyme or composition thereof as disclosed herein) is combined with one or more additional preservative substance, for example, a biocidal substance selected from polylysine, chitosan, benzoate, nisin, lysozyme, and chitosan, or any combination thereof, and the preservative (e.g., antimicrobial) effect achieved between the biobased preservative and the additional preservative substance(s) is additive or synergistic.

In some embodiments, the personal care product may include emollients. Emollients include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

Suitable antioxidants include, e.g., sulfites (e.g., sodium sulfite), tocopherol or derivates thereof, ascorbic acid or derivates thereof, citric acid, propyl gallate, chitosan glycolate, cysteine, N-acetyl cysteine plus zinc sulfate, thiosulfates (e.g. sodium thiosulfate), polyphenols glutathione, dithiothreitol (DTT), superoxide dismutase, catalase and the like.

Chelators, such as ethylene diamine tetraacetic acid (EDTA), may also be included.

Suitable thickeners include, e.g., acrylates/steareth-20 methacrylate copolymer, carbomer, carboxymethyl starch, cera alba, dimethicone/vinyl dimethicone crosspolymer, propylene glycol alginate, hydroxyethylcellulose, hydroxypropyl methylcellulose, silica, silica dimethyl silylate, xanthan gum, and hydrogenated butylenes/ethylene/styrene copolymer.

Suitable moisturizers include, e.g., butylene glycol, cetyl alcohol, dimethicone, dimyristyl tartrate, glucose glycereth-26, glycerin, glyceryl stearate, hydrolyzed milk protein, lactic acid, lactose and other sugars, laureth-8, lecithin, octoxyglycerin, PEG-12, PEG 135, PEG-150, PEG-20, PEG-8, pentylene glycol, hexylene glycol, phytantriol, poly quaternium-39 PPG-20 methyl glucose ether, propylene glycol, sodium hyaluronate, sodium lactate, sodium PCA, sorbitol, succinoglycan, synthetic beeswax, tri-C14-15 alkyl citrate, and starch.

1. Color Molecules

The compositions described herein may contain one or more color producing molecule, such as a dye or pigment molecule, for application and binding to a surface, such as binding to one or more protein on the surface of skin, such as collagen, keratin, and/or elastin, or binding to an edible casing for a food product, such as a sausage casing. Nonlimiting examples of color producing molecules are described in “Summary of Color Additives for Use in the United States in Foods, Drugs, Cosmetics, and Medical Devices,” US Food and Drug Administration, https://www.fda.gov/industry/color-additive-inventories/summary-color-additives-use-united-states-foods-drugs-cosmetics-and-medical-devices.

2. Sunscreen Molecules and Linkers

The compositions described herein may contain one or more UV-blocking molecule(s), such as a sunscreen, for application and binding to a protein or peptide within the product formulation or on the surface of skin, such as collagen, keratin, elastin, hydrolyzed collagen, hydrolyzed keratin, and/or hydrolyzed elastin.

Nonlimiting examples of sunscreen and/or sunscreen analog molecules include but are not limited to, para-aminobenzoic acid, trolamine salicylate, cinoxate, dioxybenzone, ensulizole, homosalate, meradimate, octinoxate, octisalate, octocrylene, padimate O, sulisobenzone, oxybenzone, avobenzone, and benzophenone hydrazone.

In some embodiments, the sunscreen is functionalized with a linker molecule to provide a substrate handle for enzymatic binding to a protein or peptide. A non-limiting example of this functionalization may be accomplished through formation of a Schiff base between the sunscreen molecule and linker. A non-limiting example of this functionalization may be accomplished through formation of a carbamate linkage between the sunscreen molecule and linker. The linker may include an available amine for enzyme recognition in the form of a primary amine, hydrazine, hydrazide, or alkoxyamine moiety. The linker may also include a glutamine residue for enzyme recognition. The linker may consist of two functional chemical end groups linked by an aliphatic carbon chain of varying length for in situ formation of the sunscreen-linker molecule. Nonlimiting examples of linkers include lysine, cadaverine, putrescine, hydrazine, adipic acid dihydrazide, sebacic dihydrazide, and hexamethylenediamine.

In some embodiments, the sunscreen-linker adduct is bound to a protein or peptide of interest and the sunscreen can be subsequently released by hydrolysis. In one embodiment, the sunscreen molecule is hydrolysable or otherwise releasable from the linker. In some embodiments, the sunscreen-linker adduct remains bound to a protein or peptide, e.g., a protein or peptide present on skin, to provide UV-blocking protection.

3. Proteins and Peptides

The compositions described herein may contain one or more proteins or peptides of interest for sunscreen, skin care, and/or cosmetic products or applications of use. Nonlimiting examples of proteins and peptides of interest for skin care products and cosmetics are: collagen, hydrolyzed collagen, keratin, hydrolyzed keratin, elastin, hydrolyzed elastin, silk, hydrolyzed silk, silk fibroin peptide, acetyl hexapeptide-3, acetyl hexapeptide-8, acetyl tetrapeptide-5, acetyl tetrapeptide-9, acetylarginyltryptophyl diphenylglycine, copper tripeptide-1, CT-2, dipeptide-2, heptapeptide-7, hexanoyl dipeptide-3 norleucine acetate, hexapeptide-9, hexapeptide-11, manganese tripeptide-1, myristoyl hexapeptide-16, myristoyl hexapeptide-16, myristoyl pentapeptide-17, nonapeptide-1, palmitoyl dipeptide-5 diaminobutyroyl Hydroxythreonine, palmitoyl dipeptide-5 diaminohydroxybutyrate, palmitoyl hexapeptide-12, palmitoyl hexapeptide-14, palmitoyl hexapeptide-6, palmitoyl pentapeptide-4, palmitoyl tetrapeptide-7, palmitoyl tripeptide-1, palmitoyl tripeptide-3, palmitoyl tripeptide-38, pentapeptide-3, pentapeptide-18, sh-oligopeptide-1, sh-oligopeptide-2, sh-polypeptide-1, sh-polypeptide-11, sh-polypeptide-9, soybean peptide, tetrapeptide PKEK, tetrapeptide-21, tripeptide-1, tripeptide-10 citrulline, and modified hydrolysates of hemoglobin, rice, soy, wheat protein, corn, fibronectin, reticulin, serum protein, wheat gluten.

The compositions described herein may contain one or more model peptides of interest. One non-limiting example of a model peptide of interest includes Cbz-Gln-Gly.

B. Household/Industrial Products

Non-limiting embodiments of household/industrial products which may incorporate the disclosed Tgase variant enzymes or compositions thereof as disclosed herein as a preservative substance, either alone or in combination with one or more additional preservative substance, such as one or more petrochemically derived preservative substance, include, but are not limited to, householder cleaners, such as concentrated liquid cleaners and spray cleaners, cleaning wipes, dish washing liquid, dish washer detergent, spray-mop liquid, furniture polish, indoor paint, outdoor paint, dusting spray, laundry detergent, fabric softener, rug/fabric cleaner, window and glass cleaner, toilet bowl cleaner, liquid/cream cleanser, etc. In a particular embodiment, the compositions described herein may be used in a food wash product, e.g., designed to clean fruits and vegetables prior to consumption. In some embodiments, one or more biobased preservative (i.e., Tgase variant enzyme or composition thereof as disclosed herein) is combined with one or more synthetic preservative (e.g., petrochemically derived substance) and the preservative (e.g., antimicrobial) effect achieved between the biobased and synthetic preservatives is additive or synergistic.

C. Leather

In general, a Tgase variant enzyme as described herein can be incorporated into any natural collagen containing product or used during leather processing to modify the leather such that color is covalently bound to one or more protein in leather, such as animal or non-animal derived collagen, keratin, silk, and/or elastin proteins.

D. Food Products

In general, an Tgase variant enzyme as described herein can be incorporated into any food protein or used during food processing, to modify the color of food protein. Food products into which the disclosed Tgase variant enzymes may be incorporated include, but are not limited to, animal-derived products containing collagen or gelatin (hydrolyzed collagen). These include, but are not limited to, gelatin products, meat products or meat analogue products such as sausage casings, pork rinds, or any meat or marine product including the skin layer of the animal and/or collagen. In addition, the enzyme composition may be incorporated into non-animal derived collagen-containing products or any collagen-containing product.

E. Other Products

Other products into which the disclosed Tgase variant enzymes or compositions thereof as disclosed herein may be incorporated include, but are not limited to, food, pharmaceutical, cosmetic, healthcare, marine, paint, coating, adhesive, energy (e.g., fracking fluid), plastic, packaging, and agricultural products. In some embodiments, the disclosed enzymes or enzyme-polymer compositions disclosed herein may be incorporated into HVAC systems, cooling ponds, water purification systems, or may be used in an industrial application, such as, but not limited to, pulp and paper processing.

In some embodiments, a biocidal enzyme, i.e., Tgase variant enzyme as disclosed herein, is combined with one or more additional preservative substance, such as one or more petrochemically derived preservative substance. In some embodiments, one or more biobased preservative (i.e., Tgase variant enzyme or composition thereof as disclosed herein) is combined with one or more synthetic preservative (e.g., petrochemically derived substance) and the preservative (e.g., antimicrobial) effect achieved between the biobased and synthetic preservatives is additive or synergistic.

### V. Methods of Use

Methods are provided for use of the Tgase variants disclosed herein (including any of the variants disclosed in Table 3, optionally with an N-terminal methionine residue, including circular permutants thereof, and optionally with a pro-sequence as described herein) in various applications of use in which crosslinking of proteins or peptides is desired or beneficial.

Tgase variants as described herein may be used in applications of use such as, but not limited to, preservative, antimicrobial, pharmaceutical, cosmetic, topical, industrial, energy, healthcare, or marine applications.

The Tgase variants may be employed as antimicrobial agents with applications in healthcare products, personal care or cosmetic formulations, packaging (food, cosmetic, and pharmaceuticals), textile and leather production, paints and coatings, and marine applications including water treatment and purification. The Tgase variants may be employed for permanently modifying proteins of interest, by way of example keratin and collagen, with dyes or proteins.

Non-limiting examples of methods in which the Tgase variants described herein may be used are described in PCT Application No. PCT/US20/21211, and in U.S. Provisional Application Nos. 63/010,987, 63/074,288, and 63/075,763, which are incorporated herein by reference in their entireties.

A. Preservative Methods

A Tgase variant as described herein (i.e., any of the variants disclosed in Table 3, optionally with an N-terminal methionine residue, including circular permutants thereof, and optionally with a pro-sequence as described herein) may be used as an alternative to or in addition to conventional preservatives, such as, but not limited to, parabens, formaldehyde, and glutaraldehyde and conventional biocidal agents, including silver (used in wound care products), in various applications that require preservatives for example, personal care, household, industrial, food, pharmaceutical, cosmetic, healthcare, marine, paint, coating, adhesive, energy, plastic, packaging, and agricultural products. A Tgase variant may be used as an antimicrobial (e.g., preservative) ingredient that inhibits the growth of potentially harmful bacteria, fungi, and/or other microbes, and accordingly, is added to a product to be preserved in an effective amount to inhibit bacterial, fungal, and/or microbial growth in such a products. Nonlimiting examples of such applications of use are described, for example, in PCT/US20/21211, which is incorporated by reference herein in its entirety. In some embodiments, USP <51> passing criteria are achieved, i.e., for Category 2 Products: Bacteria: No less than 2.0 log reduction from the initial calculated count at 14 days, and no increase from the 14 days' count at 28 days; for Yeast and Molds: No increase from the initial calculated count at 14 and 28 days. In some embodiments, the antimicrobial behavior of the enzymes and enzyme-biopolymer coformulations are characterized by MIC (minimum inhibitory concentration) against gram-positive and gram-negative bacteria as well as fungi, which results in reduction of microbial growth by approximately 80-100%, or any of at least about 80%, 85%, 90%, 95%, 98%, or 99% of microbial growth.

When combined with a product as described herein, e.g., a personal care, household, industrial, food, pharmaceutical, cosmetic, healthcare, marine, paint, coating, adhesive, energy, plastic, packaging, or agricultural product, or in any of the products or systems disclosed herein, e.g., in a formulation or incorporated into a product or system as a preservative, the composition may have effective broad spectrum preservation activity over a broad pH range.

In some embodiments, the method includes adding a preservative composition as described herein (e.g., a Tgase variant or a composition thereof as described herein) to a product or system, such as a personal care, household, industrial, food, pharmaceutical, cosmetic, healthcare, marine, paint, coating, adhesive, energy, plastic, packaging, or agricultural product, or in any of the products or systems disclosed herein, e.g., in a formulation or incorporated into a product or system, wherein microbial growth is decreased and/or shelf life of the product is increased in comparison to an identical product that does not contain the preservative composition. In some embodiments, no other preservative is included in the product composition, such as, but not limited to formaldehyde and/or glutaraldehyde.

In some embodiments, a method for increasing the shelf-life, integrity, or microbial free (e.g., bacterial and/or fungal free) status of a product composition, such as a personal care, household or industrial product is provided, wherein the method includes incorporating an effective amount of a preservative composition as described herein into the product (e.g., personal care, household or industrial product). In some embodiments, the effective amount may be an amount, referred to as the MIC (minimum inhibitory concentration), which results in reduction of microbial growth by approximately 80-100%, or any of at least about 80%, 85%, 90%, 95%, 98%, or 99% reduction of microbial growth as described herein.

In some embodiments of the methods or compositions described herein, the Tgase variant enzyme may be included at a concentration of about 0.01% w/v to about 5% w/v, or any of at least about 0.01% w/v, 0.05% w/v, 0.1% w/v, 0.5% w/v, 1% w/v, 1.5% w/v, 2% w/v, 2.5% w/v, 3% w/v, 3.5% w/v, 4% w/v, 4.5% w/v, or 5% w/v, or any of about 0.01% w/v to about 0.05% w/v, about 0.1% w/v to about 0.5% w/v, about 1% w/v to about 1.5% w/v, about 1.5% w/v to about 2% w/v, about 2% w/v to about 2.5% w/v, about 2.5% w/v to about 3% w/v, about 3% w/v to about 3.5% w/v, about 3.5% w/v to about 4% w/v, about 4% w/v to about 4.5% w/v, about 4.5% w/v to about 5% w/v, about 0.01% w/v to about 0.1% w/v, about 0.1% w/v to about 1% w/v, about 1% to about 5% w/v, about 0.05% w/v to about 0.5% w/v, about 0.5% w/v to about 5% w/v, about 1% w/v to about 2.5% w/v, or about 2.5% w/v to about 5% w/v.

Non-limiting examples of personal care products to which the preservative methods may be applied, utilizing the disclosed Tgase variants and compositions thereof, include bar soap, liquid soap (e.g., hand soap), hand sanitizer (including rinse off and leave-on alcohol based and aqueous-based hand disinfectants), preoperative skin disinfectant, cleansing wipes, disinfecting wipes, body wash, acne treatment products, antifungal diaper rash cream, antifungal skin cream, shampoo, conditioner, cosmetics (including but not limited to liquid or powder foundation, liquid or solid eyeliner, mascara, cream eye shadow, tinted powder, “pancake” type powder to be used dry or moistened, make up removal products etc.) deodorant, antimicrobial creams, body lotion, hand cream, topical cream, aftershave lotion, skin toner, mouth wash, toothpaste, sunscreen lotion, and baby products such as, but not limited to, cleansing wipes, baby shampoo, baby soap, and diaper cream. The present subject matter may also be applied to wound care items, such as, but not limited to, wound healing ointments, creams, and lotions, wound coverings, burn wound cream, bandages, tape, and steri-strips, and medical articles such as medical gowns, caps, face masks, and shoe-covers, surgical drops, etc. Additional products include but are not limited to oral products such as mouth rinse, toothpaste, and dental floss coatings, veterinary and pet care products, preservative compositions, and surface disinfectants including solutions, sprays or wipes.

Non-limiting examples of household/industrial products to which the preservative methods may be applied, utilizing the disclosed Tgase variants and compositions thereof, include householder cleaners such as concentrated liquid cleaners and spray cleaners, cleaning wipes, dish washing liquid, dish washer detergent, spray-mop liquid, furniture polish, indoor paint, outdoor paint, dusting spray, laundry detergent, fabric softener, rug/fabric cleaner, window and glass cleaner, toilet bowl cleaner, liquid/cream cleanser, etc. In a particular embodiment, the preservative methods of the present subject matter may be used in a food wash product, designed to clean fruits and vegetables prior to consumption, packaging, and food coatings.

B. Protein Modification Methods

In some embodiments, a Tgase variant may be included in a product to be used for long-lasting application of functional ingredients including UV-blocking sunscreens, and/or coloring agents, such as pigments or dyes. For example, the Tgase variant may be used in a composition for delivery of an active or functional ingredient to mammalian (e.g., human) skin, hair, or nails, such as, but not limited to, permanent (covalent) color modification of the surface of hair fibers. In some embodiments, the Tgase variant may be incorporated in a product to be applied topically and which bonds to the skin of an individual, such as a UV-blocking (sunscreen) product, or a cosmetic product. In some embodiments, the Tgase variant may be used to provide permanent application of color to the skin of an animal such as in leather processing. In some embodiments, the Tgase variant may be used to provide a permanent application of color in food processing.

Methods are provided herein for modifying or adding color to a protein or material of interest. The methods include contacting a protein, peptide, or material of interest with one or Tgase variant as described herein and one or more functional ingredient including a sunscreen and/or color-producing molecule, e.g., a dye or pigment molecule. The Tgase variant enzyme(s) are present in an amount that is sufficient (i.e., effective) to covalently bind the sunscreen and/or color molecule(s) to the protein, peptide, or material of interest.

In some embodiments, the protein of interest is one or more protein present in skin, and the Tgase enzyme(s) and sunscreen(s) and/or color molecule(s) may be in the form of a cosmetic or personal care product. The protein present in skin may be collagen, keratin, and/or elastin.

In some embodiments, the material of interest is one or more protein or peptide derived from skin, and the Tgase variant enzyme(s) and/or sunscreen(s) and/or color molecule(s) may be in the form of a cosmetic or personal care product. The protein present in the product formulation may be collagen, keratin, and/or elastin. The peptide present in the product formulation may be hydrolyzed collagen, hydrolyzed keratin, and/or hydrolyzed elastin.

In some embodiments, the protein or material of interest is leather, a food product, or an agricultural product, or a protein of interest therein, and the Tgase variant enzyme(s) and/or color molecule(s) are in the form of a composition that is suitable for modifying or adding color to the leather, a food product, or an agricultural product, or a protein of interest therein.

In some embodiments, a method is provided for delivering an active or functional ingredient (such as a sunscreen molecule or coloring agent) to proteins or peptides of mammalian (e.g., human) skin, hair, or nails. For example, the method may include application of a composition as described herein to proteins or peptides of mammalian (e.g., human) skin, hair, or nails or topical application of the composition to skin, hair, or nails of a mammalian (e.g., human) individual.

In some embodiments, the method includes contacting proteins and/or peptides of mammalian (e.g., human) skin, hair, or nails, with a composition that includes: (a) an effective amount of at least one active or functional ingredient (such as, for example, a sunscreen molecule or coloring agent); and (b) a Tgase variant enzyme in an amount effective to catalyze the crosslinking of the active or functional ingredient to a protein or peptide of mammalian (e.g., human) skin, hair, or nails. In some embodiments, the method includes topical application of the composition to the skin, hair, or nails of a mammalian (e.g., human) individual, and in certain embodiments the composition may contain: (c) a pharmaceutically or acceptable carrier in an amount effective to deliver the Tgase variant enzyme and the active or functional ingredient to the skin, hair, or nails of the individual.

For example, the active or functional ingredient may include at least one alkylamino (—RNH₂), hydrazine, hydrazide, or hydroxylamine moiety, either directly on the active or functional ingredient, or indirectly on a linker attached (e.g., covalently bound) thereto, and the method includes catalysis by the transglutaminase enzyme of crosslinking (e.g., formation of covalent bonds) between the amino groups of the active or functional ingredient and amino groups (e.g., amino groups on glutamine and/or lysine amino acid residue side chains) in proteins or peptides of skin, hair, or nails.

### EXAMPLES

The following examples are intended to illustrate, but not limit, the invention.

### Example 1

A site saturation library of mature transglutaminase (SEQ ID NO: 1) was synthesized to create single point mutant enzyme variants. In an effort to create enzyme variants with rapid kill rates and longevity in product formulations, variants were identified that demonstrate activity in an endpoint assay in addition to identifying enhancements in initial rates of activity.

The DNA variants were synthesized by standard methods, and the enzyme was expressed. The resulting single point mutation enzyme variants were assayed using a primary screen, i.e., the standard colorimetric hydroxamate activity assay for Tgase (Folk and Cole (1965) J Biol Chemistry 240(7):2951-60), and a secondary screen was performed to determine initial activity rates of the active mutants using transglutaminase-catalyzed labeling of casein with dansylcadaverine (e.g., a commercially available kit such as the Transglutaminase Fluorogenic Activity Assay Kit, T036, Zedira, Germany).

Briefly, the standard hydroxamate assay uses N-carbobenzoxy-L-glutaminylglycine (Z-Gln-Gly or CBZ-Gln-Gly) as the amine acceptor substrate and hydroxylamine as an amine donor. In the presence of transglutaminase, the hydroxylamine is incorporated to form Z-glutamylhydroxamate-glycine which develops a colored complex with iron (III), detectable at 525 nm after incubation at 37° C. for 1-3 hours. The calibration was performed using L-glutamic acid γ-monohydroxamate (Millipore Sigma) as standard. One unit of Tgase is defined as the amount of enzyme that catalyzes formation of 1 μmol of the peptide derivative of γ-glutamylhydroxylamine per minute.

The Transglutaminase Fluorogenic Activity Assay Kit monitors the transglutaminase-catalyzed covalent coupling of monodansylcadaverine into N,N-dimethylcasein, which produces a shift in intensity and wavelength of fluorescence of the dansyl group. The transglutaminase activity can be monitored online by measurement of the fluorescence (excitation wavelength 332 nm; emission wavelength 500 nm). The relative transglutaminase activity is shown by increase of fluorescence intensity overtime.

Each variant was evaluated and ranked for improvements in activity relative to wild-type Tgase. Variants with improved activity of at least about 1.4-fold (40%) in either assay were identified and the mutations of these variants are shown in Table 3. The amino acid positions of mutations depicted in Table 3 are relative to the wild-type Streptomyces mobaraensis mature Tgase sequence depicted in SEQ ID NO:1.

TABLE 3
Table 1. Streptomyces mobaraensis Tgase variants and
improvements to activity relative to wild-type
S. mobaraensis Tgase. Numbering of amino acid positions
is in reference to the mature S. mobaraensis Tgase amino
acid sequence depicted in SEQ ID NO: 1.

| | Activity |
|---|---|
| Variant | Improvement |
| A10C | + |
| A10Q | ++ |
| D14H | + |
| D14L | + |
| D14M | + |
| D14N | ++ |
| D14W | + |
| D14Y | + |
| R15A | + |
| R15E | + |
| R15T | + |
| D18E | + |
| D18T | + |
| G47H | ++ |
| R48M | + |
| K49E | ++ |
| K49T | ++ |
| Q74C | + |
| N134S | + |
| N134T | + |
| A136C | + |
| A136S | + |
| L137K | +++ |
| L137V | + |
| L147E | + |
| L147M | + |
| E164F | + |
| P169E | + |
| F170I | ++ |
| F170L | + |
| F170V | ++ |
| S199A | ++ |
| S199G | + |
| S299A | + |
| S299E | + |
| S299K | + |
| S299V | ++ |

An improvement in enzyme activity of 1.4-fold or greater is denoted by “+”; an improvement of 1.6-fold or greater is denoted by “++”; an improvement of 1.8-fold or greater is denoted by “+++”.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated in the appended claims.

All publications, patents, and patent applications cited herein are hereby incorporated by reference in their entireties for all purposes and to the same extent as if each individual publication, patent, or patent application were specifically and individually indicated to be so incorporated by reference.

We claim:

1. A transglutaminase enzyme comprising or consisting of an amino acid sequence selected from the sequences depicted in Table 3, optionally further comprising an N-terminal methionine residue.

2. The transglutaminase enzyme of claim 1, further comprising a pro-sequence.

3. A transglutaminase enzyme that is a variant of the mature transglutaminase enzyme of Streptomyces mobaraensis, selected from A10C or Q, D14H, L, M, N, W, or Y, R15A, E, or T, D18 E or T, G47H, R48M, K49E or T, Q74C, N134S or T, A136C or S, L137K, V, E, or M, E164F, P169E, F170I, L, or V, S199A or G, and S299A, E, K, or V, optionally further comprising an N-terminal methionine residue.

4. The transglutaminase enzyme of claim 3, further comprising a pro-sequence.

5. A transglutaminase enzyme comprising a circular permutant of any of the amino acid sequences depicted in Table 3, optionally further comprising an N-terminal methionine residue.

6. The transglutaminase enzyme of claim 5, further comprising a pro-sequence.

7. A method for increasing the shelf life of a product, comprising incorporating the transglutaminase variant of any of claims 1 to 6 into the product in an amount effective to prevent or decrease growth of one or more microbe in comparison to an identical product that does not comprise the composition.

8. A product comprising the transglutaminase enzyme of any of claims 1 to 6 in an effective amount to increase the shelf life of the product, in comparison to an identical product that does not comprise the enzyme.

9. The product of claim 8, wherein the product is a personal care, household, industrial, food, pharmaceutical, cosmetic, healthcare, marine, paint, coating, energy, plastic, packaging, or agricultural product.

10. The product of 9, wherein the product is selected from bar soap, liquid soap, hand sanitizer, preoperative skin disinfectant, cleansing wipes, disinfecting wipes, body wash, acne treatment products, antifungal diaper rash cream, antifungal skin cream, shampoo, conditioner, cosmetics deodorant, antimicrobial creams, body lotion, hand cream, topical cream, aftershave lotion, skin toner, mouth wash, toothpaste, and sunscreen lotion.

11. The product of claim 9, wherein the product is a wound care product selected from wound healing ointments, creams, and lotions, wound coverings, burn wound cream, bandages, tape, and steri-strips.

12. An enzyme composition comprising: (i) the transglutaminase enzyme of any of claims 1 to 6; and (ii) a substrate for the transglutaminase enzyme, comprising a sunscreen molecule, a pigment, or a dye molecule.

13. The enzyme composition of claim 12, wherein the sunscreen molecule, pigment or dye molecule is conjugated to a molecule that comprises a free amino group.

14. The enzyme composition of claim 13, wherein the molecule that comprises a free amino group is selected from lysine, cadaverine, putrescine, hydrazine, adipic acid dihydrazide, sebacic dihydrazide, and hexamethylenediamine.

15. The enzyme composition of claim 12, wherein the sunscreen molecule, pigment, or dye molecule is conjugated to an amino acid, peptide, or protein with a free glutamine side chain.

16. A cosmetic composition comprising the enzyme composition of claim 12.

17. A method for bonding color to a material or protein of interest, comprising contacting the material or protein of interest with the transglutaminase enzyme of any of claims 1 to 6 and a pigment or dye molecule, wherein the transglutaminase enzyme is present in an amount effective to covalently bind the pigment or dye molecule to the material or protein of interest.

18. The method of claim 17, wherein the protein of interest is a protein that is present in skin.

19. The method of claim 17, wherein the protein that is present in skin comprises collagen, keratin, and/or elastin.

20. A product comprising the transglutaminase enzyme of any of claims 1 to 6 in an effective amount to add a color molecule onto a protein or a protein-, peptide-, or amino acid-containing material of interest when contacted with the product.

21. The product of claim 20, wherein the product is a personal care, cosmetic, leather, food, or agricultural product.

22. A method of modifying the color of a protein or material of interest, comprising contacting the protein or material of interest with the product according to claim 21.

23. A composition comprising the transglutaminase enzyme of any of claims 1 to 6 in combination with one or more antimicrobial enzyme, peptide, or protein, wherein the composition comprises preservative, biocidal, antimicrobial, or virucidal activity.

24. The composition of claim 23, wherein the antimicrobial enzyme, peptide, or protein is selected from lysozyme, chitinase, lipase, lysin, lysostaphin, glucanase, DNase, RNase, lactoferrin, glucose oxidase, peroxidase, lactoperoxidase, lactonase, acylase, dispersin B, a-amylase, cellulase, nisin, bacteriocin, siderophore, polymyxin, and defensin.

25. A bacteriophage, comprising a nucleic acid sequence that encodes the transglutaminase enzyme of any of claims 1 to 6.

26. The composition of claim 25, wherein the composition comprises antimicrobial activity.

27. The composition of claim 26, wherein the composition further comprises a pharmaceutically acceptable excipient.
